(19) United States
(12) Patent Application Publication
(10) Pub. No.: US 2007/0092502 A1
(43) Pub. Date: Apr. 26, 2007

(54) Method of Treating Glaucoma

(75) Inventor: Martin A. Voet, San Juan Capistrano, CA (US)

Correspondence Address: ALLERGAN, INC., 2525 DUPONT DRIVE, T2-7H, IRVINE, CA, 92612-1599, US

(73) Assignee: Allergan, Inc.

(21) Appl. No.: 11/426,407

(22) Filed: Jun. 26, 2006

Related U.S. Application Data
(60) Provisional application No. 60/713,794, filed on Sep. 1, 2005.

(51) Int. Cl. A61K 38/54; A61K 38/44; A61K 31/70; A61K 31/65; A61K 31/43; A61K 31/4439; A61K 31/7034

(52) U.S. Cl. 424/94.3; 424/94.4; 514/23; 514/338; 514/154; 514/192; 514/35; 424/653

(57) ABSTRACT

The present invention provides a method of treating glaucoma or preventing glaucoma in a person at risk of developing glaucoma, by applying to the eye of said person, an effective amount of an antibacterial agent having activity against the Heliocobacter Pylori bacteria to thereby eradicate, inhibit and/or control said bacteria.

### CROSS REFERENCE TO RELATED APPLICATIONS

This application is based on, and claims the benefit of, U.S. Provisional Application No. 60/713,794, filed Sep. 1, 2005, and which is incorporated herein by reference.

### BACKGROUND OF THE INVENTION

1. Field of the Invention

The present invention relates to the treatment of glaucoma with inhibitors of Helicobacter pylori.

2. Description of the Related Art

It has been reported that levels of antibodies to Helicobacter pylori were found to be significantly higher in people with primary open-angle glaucoma and exfoliation glaucoma compared to cataract surgery patients in a prospective study. The authors of this study were reported to “believe that the bacteria may play a role in the pathobiology of these forms of glaucoma.” However, it was also stated that “(f)uture studies in larger glaucoma cohorts throughout the clinical range are needed to verify these findings . . . ”

Since Helicobacter pylori has been implicated in causing gastritis and peptic ulcers and may be implicated in causing stomach cancer, various pharmaceutically-active compounds have been disclosed as useful for preventing and/or treating the conditions caused by the bacteria. These compounds are generally suitable for eradicating the causative bacteria in the gastrointestinal tract.

For example, U.S. Pat. No. 6,489,317 discloses that the combination of ansamycin and a second antibiotic or antimicrobial agent may be used to treat and/or prevent the reoccurrence of a gastrointestinal disorder associated with Helicobacter pylori.

U.S. Pat. No. 6,149,908 discloses an antibacterial system comprising lactoperoxidase and a peroxide donor for preparing a prophylactic or therapeutic treatment, in vivo, of infections caused by Helicobacter pylori existing in the stomach of a patient. This preparation also includes a thiocyanate and lactoferrin.

U.S. Pat. No. 6,555,534 discloses that 4,4-methylenebis(tetrahydro-1,2-4-thiadiazine-1,1-dioxide) may be used to eradicate and control Helicobacter pylori in the luminal mucosal surface of the stomach and duodenum of a patient.

A composition including a protease and an antibacterial agent is disclosed for removing Helicobacter pylori from the stomach of a patient without causing side effects or the occurrence of resistant bacteria. (See U.S. Pat. No. 5,618,564.)

Quinolones are disclosed as useful for treating gastroduodenal disorders, diseases and adverse conditions caused by Helicobacter pylori. (See U.S. Pat. No. 5,942,619.)

Certain nitrothiazole compounds such as

(2-(acetolyloxy)-N-(5-nitro 2-thiazoyl) benzamide

may be used for treating diseases or infections due to Helicobacter pylori bacteria.

Finally, in the following patents additional compounds are disclosed as useful for treating and/or preventing disorders caused by Helicobacter pylori bacteria.

| U.S. Pat. No. | Active Compound |
|---|---|
| 5,750,535 | Substance P receptor antagonists |
| 6,028,062 | Dimeticone |
| 6,444,703 | [(4-[4-(4-methylbenzyloxycarbonyl) phenyl[phenyl |
| | trans-4-guanidinomethylcyclohexanecarboxylate or an |
| | acid addition salt |

All of the above patents are hereby incorporated by reference to disclose compounds useful in the method of the present invention.

In U.S. Pat. Nos. 6,416,968 and 6,762,051 disclose methods of screening molecules that inhibit the survival of Helicobacter pylori bacteria. These methods may be utilized to obtain additional compounds useful in the method of the present invention.

### BRIEF SUMMARY OF THE INVENTION

The present invention provides a method of treating glaucoma or preventing glaucoma in a person at risk of developing glaucoma, by applying to the eye of said person, an effective amount of an antibacterial agent having activity against the Heliocobacter Pylori bacteria to thereby eradicate, inhibit and/or control said bacteria.

### DETAILED DESCRIPTION OF THE INVENTION

The antibacterial agent utilized in the method of the present invention may be any compound, combination of compounds, composition, etc. (which combination may be administered in a single composition or the individual compounds of said combination may be administered serially), that is effective to control, inhibit and/or eradicate the Heliocobacter Pylori bacteria when delivered to the eye of a patient having glaucoma or at risk of developing glaucoma.

For example, the antibacterial agent may be lactoperoxidase and a peroxide donor which may be administered in accordance with the teaching of U.S. Pat. No. 6,149,908. The antibacterial agent may be the “Lactoperoxidase system” which is disclosed in said U.S. patent as including 50 mg/l lactoperoxidase (25 U/mg); 4.5 g/l glucose; 6.1 mg/l glucoseoxidase (200 U/mg); 35 mg/l thiocyanate.

The antibacterial agent may be a combination of ansamycin and another antibiotic, as selected in accordance with U.S. Pat. No. 6,489,317, in an amount as disclosed in said patent. For example, the antibacterial agent may comprise, rifabutin and a therapeutically effective amount of a second antibiotic or antimicrobial agent selected from the group consisting of amoxicillin, tetracycline and bismuth compounds. To this combination may be added a therapeutically effective amount of a proton pump inhibitor selected from the group consisting of omeprazole, pantoprazole, rabeprazole and lansoprazole.

Alternatively, as disclosed in U.S. Pat. No. 5,618,564, the antibacterial agent may be the combination of a protease, e.g. pronase, trypsin, α-chymotrypsin, serrapeptase, bromelain and pepsin and an antibacterial agent, e.g. an antibiotic, an anti-protozoan drug or a bismuth preparation. Specific antibiotics, anti-protozoan drugs and bismuth preparations include amoxicillin, erythromycin and clindamycin; metronidazole and tinidazole; and bismuth, bismuth subnitrate, bismuth subsalicylate and colloidal bismuth, respectively.

Another suitable antibacterial agent may be selected from the quinoline compounds disclosed in U.S. Pat. No. 5,942,619. That is, the antibacterial agent may be one or more compound selected from the group consisting

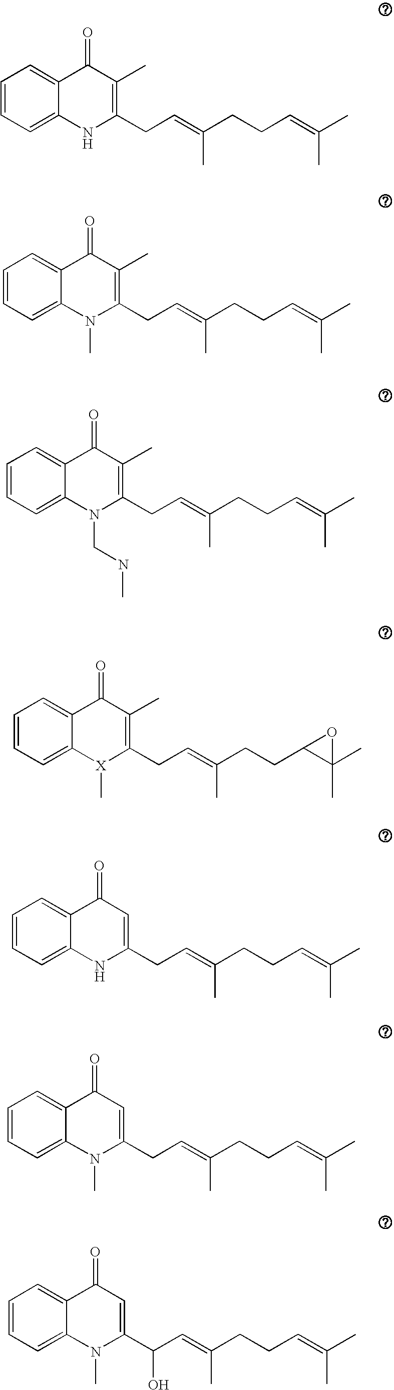

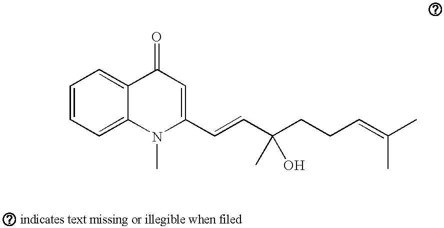

The antibacterial agent may be selected from the group of substance P receptor antagonists disclosed in U.S. Pat. No. 5,750,535, For example, these substant P receptor antagonists may be selected from the group consisting of
(2S,3S)—N-(5-isopropyl-2-methoxyphenyl)methyl-2-diphenylmethyl-1-azabicyclo-[2.2.2]octan-3-amine;
(2S,3S)—N-(5-tert-butyl-2-methoxyphenyl)methyl-2-diphenylmethyl-1-azabicyclo-[2.2.2]octan-3-amine;
(2S,3S)—N-(5-methyl-2-methoxyphenyl)methyl-2-diphenyl-methyl-1-azabicyclo-[2.2.2]octan-3-amine;
(2S,3S)—N-(5-ethyl-2-methoxyphenyl)methyl-2-diphenyl-methyl-1-azabicyclo-[2.2.2]octan-3-amine;
(2S,3S)—N-(5-isopropyl-2-methoxyphenyl)methyl-2-diphenylmethyl-1-azabicyclo-[2.2.2]octan-3-amine;
(2S,3S)—N-(5-sec-butyl-2-methoxyphenyl)methyl-2-diphenylmethyl-1-azabicyclo-[2.2.2]octan-3-amine; and
(2S,3S)—N-(5-n-propyl-2-methoxyphenyl)methyl-2-diphenylmethyl-1-azabicyclo-[2.2.2]octan-3-amine, and the pharmaceutically acceptable salts of the foregoing compounds.

The compounds disclosed in U.S. Pat. No. 6,444,703 may be utilized in the method of the present invention. That is, [4-[4-(4-methylbenzyloxycarbonyl)phenyl[phenyl trans-4-guanidinomethylcyclohexanecarboxylate or an acid addition salt thereof is a suitable antibacterial agent for use in the method of the invention.

The method of the present invention may use as said antibacterial agent a compound selected from the group consisting of

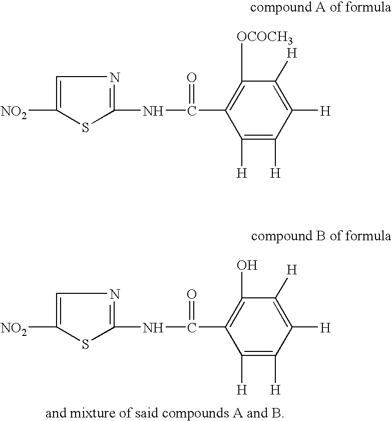

(Also, see U.S. Pat. No. 6,555,534 wherein 4,4-methylenebis(tetrahydro-1,2-4-thiadiazine-1,1-dioxide may be used in the method of the present invention.)

Finally, the method of the present invention may utilize dimethicone in the local treatment of glaucoma. (See U.S. Pat. No. 6,028,062.)

For treatment of diseases affecting the eye including glaucoma, these compounds can be administered topically, periocularly, intraocularly, or by any other effective means known in the art. The compounds disclosed herein may be administered topically, periocularly, or by intraocular injection. Delivery may be by sustained release. For example, the drug may be delivered via a sustained release polymer, where the drug is released over time by diffusion of the drug from the polymer or degradation of the polymer. The polymer might be injected or implanted anywhere in or around the eye, including the subconjunctival or subtenons space.

Pharmaceutical compositions may be prepared by combining a therapeutically effective amount of at least one compound according to the present invention, or a pharmaceutically acceptable acid addition salt thereof, as an active ingredient, with conventional ophthalmically acceptable pharmaceutical excipients, and by preparation of unit dosage forms suitable for topical ocular use. The therapeutically efficient amount will vary with the activity of the selected antibacterial agent; however, typically between about 0.0001 and about 5% (w/v), preferably about 0.001 to about 1.0% (w/v) of said antibacterial agent will be included in liquid formulations.

For topical ophthalmic application, preferably solutions are prepared using a physiological saline solution as a major vehicle. The pH of such ophthalmic solutions should preferably be maintained between 6.5 and 7.2 with an appropriate buffer system. The formulations may also contain conventional, pharmaceutically acceptable preservatives, stabilizers and surfactants.

Preferred preservatives that may be used in the pharmaceutical compositions of the present invention include, but are not limited to, benzalkonium chloride, chlorobutanol, thimerosal, phenylmercuric acetate and phenylmercuric nitrate. A preferred surfactant is, for example, Tween 80. Likewise, various preferred vehicles may be used in the ophthalmic preparations of the present invention. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar vein, an ophthalmically acceptable antioxidant for use in the present invention includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. The preferred chelating agent is edentate disodium, although other chelating agents may also be used in place or in conjunction with it.

The ingredients may be used in the following amounts:

| Ingredient | Amount (% w/v) |
|---|---|
| active ingredient | about 0.001-5 |
| preservative | 0-0.10 |
| vehicle | 0-40 |
| tonicity adjustor | 1-10 |
| buffer | 0.01-10 |
| pH adjustor | q.s. pH 4.5-7.5 |
| antioxidant | as needed |
| surfactant | as needed |
| purified water | as needed to make 100% |

The actual dose of the active compounds of the present invention depends on the specific compound, and on the condition to be treated; the selection of the appropriate dose is well within the knowledge of the skilled artisan.

The ophthalmic formulations of the present invention are conveniently packaged in forms suitable for metered application, such as in containers equipped with a dropper, to facilitate the application to the eye. Containers suitable for dropwise application are usually made of suitable inert, non-toxic plastic material, and generally contain between about 0.5 and about 15 ml solution.

Those skilled in the art will readily understand that for oral or rectal administration the compounds of the invention are admixed with pharmaceutically acceptable excipients which per se are well known in the art. Specifically, a drug to be administered systemically, it may be confected as a powder, pill, tablet or the like, or as a syrup or elixir suitable for oral administration. Description of the substances normally used to prepare tablets, powders, pills, syrups and elixirs can be found in several books and treatise well known in the art, for example in Remington's Pharmaceutical Science, Edition 17, Mack Publishing Company, Easton, Pa.

Parenteral administration is generally characterized by injection. Injectables can be prepared in conventional forms, either as liquid solutions or suspensions, solid forms suitable for dissolving or suspending in liquid prior to injection, or as emulsions. Descriptions of substances and methods normally used to prepare formulations for parenteral administration can be found in several treatises and books well known in the art such as, Handbook On Injectable Drugs (11th edition), edited by Lawrence A. Trissel, (Chicago: Login Brothers Book Company; Jan. 15, 2001).

The invention is further illustrated by the following example which is illustrative of a specific mode of practicing the invention and is not intended as limiting the scope of the claims.

### EXAMPLE

A male patient, 37 years old, who is suffering from glaucoma is found to be infected with H. pylori bacteria which is believed by the physician to be at least one of the causes of his glaucoma. The patient is treated by administration of 4 times daily doses of rifabutin, pantoprazole and tetracycline in amounts of 600 mg, 160 mg and 2000 mg per day respectively. After a period of 8 days on this treatment, the H. pylori infection in the patient is eradicated. Thereafter, it is observed by the physician that the patient's glaucoma symptoms are improved.

The foregoing description details specific methods and compositions that can be employed to practice the present invention, and represents the best mode contemplated. However, it is apparent for one of ordinary skill in the art that further compounds with the desired pharmacological properties can be prepared in an analogous manner, and that the disclosed compounds can also be obtained from different starting compounds via different chemical reactions. Similarly, different pharmaceutical compositions may be prepared and used with substantially the same result. Thus, however detailed the foregoing may appear in text, it should not be construed as limiting the overall scope hereof; rather, the ambit of the present invention is to be governed only by the lawful construction of the appended claims.

I claim:

1. A method of treating glaucoma or preventing glaucoma in a person at risk for developing glaucoma, by applying to the eye of said person, an effective amount of an antibacterial agent having activity against the Heliocobacter Pylori bacteria to thereby eradicate, inhibit and/or control said bacteria.

2. The method of claim 1 wherein antibacterial agent is a combination of lactoperoxidase and a peroxide donor.

3. The method of claim 2 wherein said combination comprises 50 mg/l lactoperoxidase (25 U/mg); 4.5 g/l glucose; 6.1 mg/l glucoseoxidase (200 U/mg); and 35 mg/l thiocyanate.

4. The method of claim 1 wherein said antibacterial agent is a combination of ansamycin and another antibiotic.

5. The method of claim 4 wherein said antibacterial agent is rifabutin and a therapeutically effective amount of a second antibiotic or antimicrobial agent selected from the group consisting of amoxicillin, tetracycline and bismuth compounds.

6. The method of claim 5 wherein said antibacterial agent further comprises a proton pump inhibitor.

7. The method of claim 6 wherein said proton pump inhibitor is selected from the group consisting of omeprazole, pantoprazole, rabeprazole and lansoprazole.

8. The method of claim 1 wherein said antibacterial agent is a combination of a protease, and an antibacterial agent.

9. The method of claim 8 wherein said protease is selected from the group consisting of pronase, trypsin, α-chymotrypsin, serrapeptase, bromelain and pepsin and said antibacterial agent is selected from the group consisting of an antibiotic, an anti-protozoan drug and a bismuth preparation.

10. The method of claim 9 wherein said antibiotic is selected from the group consisting of amoxicillin, erythromycin and clindamycin said anti-protozoan drug is selected from the group consisting of metronidazole and tinidazole and said bismuth preparation is selected from the group consisting of bismuth, bismuth subnitrate, bismuth subsalicylate and colloidal bismuth.

11. The method of claim 1 wherein antibacterial agent is a quinoline compound.

12. The method of claim 11 wherein said quinoline compound is selected from the group consisting of compounds represented by the formula

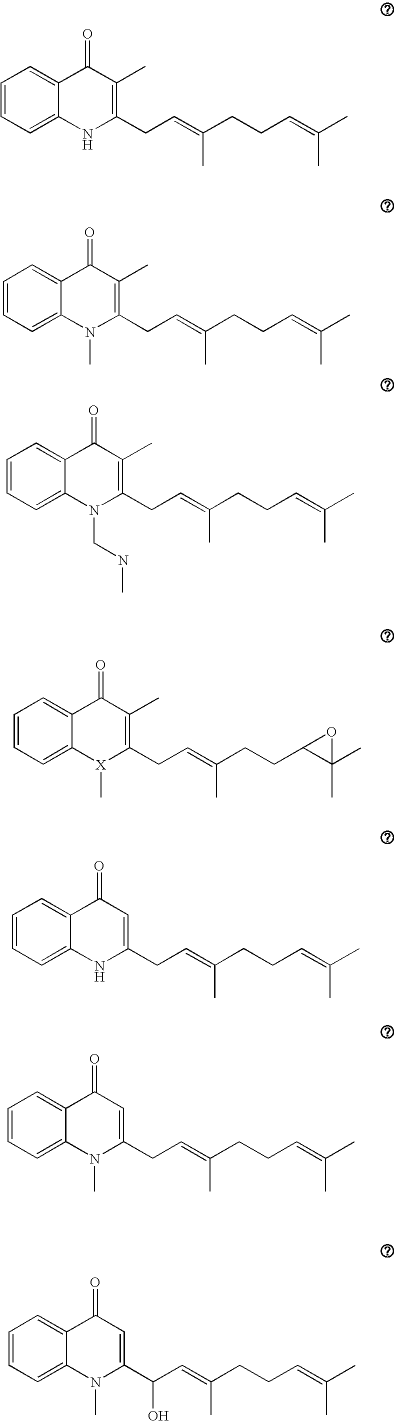

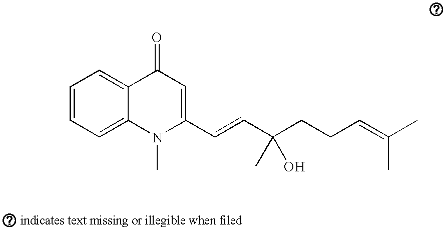

13. The method of claim 1 wherein said antibacterial agent is a substance P receptor antagonist.

14. The method of claim 13 wherein said substance P receptor antagonist is selected from the group consisting of
(2S,3S)—N-(5-isopropyl-2-methoxyphenyl)methyl-2-diphenylmethyl-1-azabicyclo-[2.2.2]octan-3-amine;
(2S,3S)—N-(5-tert-butyl-2-methoxyphenyl)methyl-2-diphenylmethyl-1-azabicyclo-[2.2.2]octan-3-amine;
(2S,3S)—N-(5-methyl-2-methoxyphenyl)methyl-2-diphenyl-methyl-1-azabicyclo-[2.2.2]octan-3-amine;
(2S,3S)—N-(5-ethyl-2-methoxyphenyl)methyl-2-diphenyl-methyl-1-azabicyclo-[2.2.2]octan-3-amine;
(2S,3S)—N-(5-isopropyl-2-methoxyphenyl)methyl-2-diphenylmethyl-1-azabicyclo-[2.2.2]octan-3-amine;
(2S,3S)—N-(5-sec-butyl-2-methoxyphenyl)methyl-2-diphenylmethyl-1-azabicyclo-[2.2.2]octan-3-amine; and
(2S,3S)—N-(5-n-propyl-2-methoxyphenyl)methyl-2-diphenylmethyl-1-azabicyclo-[2.2.2]octan-3-amine, and the pharmaceutically acceptable salts of the foregoing compounds.

15. The method of claim 1 wherein said antibacterial agent is [4-[4-(4-methylbenzyloxycarbonyl)phenyl[phenyl trans-4-guanidinomethylcyclohexanecarboxylate or an acid addition salt thereof.

16. The method of claim 1 wherein said antibacterial agent is selected from the group consisting of

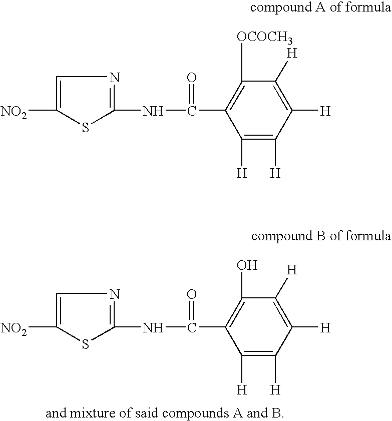

17. The method of claim 1 wherein said antibacterial agent is 4,4-methylenebis (tetrahydro-1,2-4-thiadiazine-1,1-dioxide

18. The method of claim 1 wherein said antibacterial is dimethicone.
